(19) United States
(12) Patent Application Publication
(10) Pub. No.: US 2025/0115894 A1
(43) Pub. Date: Apr. 10, 2025

(54) Three-dimensional model of exposed microbial membranes at gas-liquid interface and preparation method thereof

(71) Applicant: FUDAN UNIVERSITY, Shanghai (CN)

(72) Inventors: Dan LI, Shanghai (CN); Shuo YANG, Shanghai (CN); Xiang ZHANG, Shanghai (CN); Jianmin CHEN, Shanghai (CN)

(73) Assignee: FUDAN UNIVERSITY, Shanghai (CN)

(21) Appl. No.: 18/885,576

(22) Filed: Sep. 14, 2024

(30) Foreign Application Priority Data
Sep. 17, 2023 (CN) 202311199063X
Sep. 17, 2023 (CN) 2023112024538

(51) Int. Cl. C12N 11/10; C12N 11/04; C12N 11/084

(52) U.S. Cl. CPC C12N 11/10; C12N 11/04; C12N 11/084

(57) ABSTRACT

A rapidly constructed three-dimensional model of a microbial film exposed at an air-liquid interface and a preparation method thereof, as well as a hydrogel bead containing microorganisms immobilized at an air-liquid interface in an exposed state and a preparation method thereof. The model includes a hydrogel material containing gradually releasable nutrients, as a core scaffold; a hydrogel film attached to a surface of core and wrapping microorganisms, as a biofilm-like film; and an antimicrobial polyelectrolyte layer between the biofilm-like film structure and the core structure which allows substances in the core to be released into the biofilm-like film structure while preventing the microorganisms on the film from migrating to the core material, ensuring uniform distribution of the microorganisms on the surface of the core and maintaining the microorganisms continuously exposed at the air-liquid interface.

### FIELD OF TECHNOLOGY

The present disclosure relates to the technical field of microorganisms, specifically to a rapidly constructed three-dimensional model of a microbial film exposed at an air-liquid interface and a preparation method thereof, as well as a hydrogel bead containing microorganisms immobilized at an air-liquid interface and a preparation method thereof.

### BACKGROUND

In vitro microbial biofilm models refer to the simulation of biofilm structures created by microorganisms in natural environments under laboratory circumstances, using microbial cells and extracellular matrices on specific surfaces or scaffolds. These models may be used in various aspects, including microbial ecology, the formation mechanism of biofilms, microbial interactions, and the microbiological effects of medications. Briefly, after selecting a single microorganism or a complex microbial community, the selected microorganisms are cultivated in a medium containing a scaffold material, with necessary nutrients and growth conditions provided to support their development and proliferation. During the development and proliferation, the microorganisms gradually attach to the scaffold material under the influence of the extracellular matrices, forming a film-like structure. In vitro microbial biofilm models allow for the introduction of numerous microbial species to investigate their interactions and enable the co-culturing of biofilms with other free microorganisms to observe their synergistic effects, competition, and symbiotic connections. Furthermore, such models possess high controllability and repeatability, allowing researchers to investigate biofilm-related issues without affecting the natural environment.

Traditionally, biofilms are generated by depending on microorganisms' spontaneous biological activity. Artificially manufactured matrices may be used to aid microbial adhesion and development of microorganisms on the scaffold material, hence speeding up or improving biofilm formation and structure. However, the biofilm generation process in typical in vitro microbial biofilm models may take several days or even dozens of days, which is undeniably time-consuming. Furthermore, certain microbes are unable to naturally adhere to and develop biofilms on the scaffold material. Although in vitro models of microorganism-formed biofilms may be controlled to some degree, inherent structural variances will remain owing to spontaneous development over repeated studies. Most notably, all existing in vitro microbial biofilm models must be submerged in a culture medium to ensure proper biofilm development. Furthermore, most bacteria lack the adhesion capabilities of human epithelial cells and cannot be quantitatively exposed to air. These issues limit laboratory-based investigations of microbial behaviors in air, as well as the use of in vitro microbial biofilms in scientific research.

### SUMMARY

The present disclosure provides a rapidly constructed three-dimensional model of a microbial film exposed at an air-liquid interface and a preparation method thereof, which enables the rapid immobilization of any culturable microorganisms within minutes to form a biofilm-like state exposed at the air-liquid interface, and provides support for research on microbial behaviors, microbial interactions, drug interactions, gaseous drug administration, sterilization, and microbial air-exposure behaviors.

The rapidly constructed three-dimensional model of a microbial film exposed at an air-liquid interface according to the present disclosure, also known as an in vitro microbial model of biofilm-like film, has a multi-layered structure. Specifically, the three-dimensional model includes: a core, configured to be a hydrogel material containing gradually releasable and serving as a scaffold for carrying other structures, whose three-dimensional structure and volume can be arbitrarily controlled as required; a biofilm-like film, configured to be a layer of highly biocompatible hydrogel film attached to a surface of the core and wrapping microorganisms, enabling the microorganisms to form a biofilm-like film structure on the surface of the scaffold and survive and proliferate normally. After being blown with pure air for a certain time, due to the differences in swelling and dehydration rates between the core material and the surface hydrogel film material, the biofilm-like film can also shrink into a thin film, compelling the microorganisms to inhabit a unique air-liquid interface state. A polyelectrolyte layer, configured to be an extremely thin antimicrobial polyelectrolyte layer between the biofilm-like film structure and the core structure, allows substances in the core to be released into the biofilm-like film structure while preventing the microorganisms on the film from migrating to the core material with richer nutrients. This ensures the uniform distribution of the microorganisms on the surface of the core and maintains the microorganisms continuously exposed at the air-liquid interface. The microorganisms surviving on the biofilm-like film can receive nutrients stably released from the core scaffold from an inner surface of the film, so that the microorganisms on the biofilm-like film can be detached from a culture medium and exposed to the air, and still survive and maintain normal biological functions. In addition, the core containing nutrients is 5-2,000 times larger in volume than the biofilm-like film structure (specifically determined by the three-dimensional structure and volume of the core), that is, compared to a microorganism solution state, the microorganisms on the biofilm-like structure can access nutrients equivalent to 5 to 2,000 times their volume, and aerobic microorganisms can obtain a larger amount of oxygen at the air-liquid interface. Therefore, when exposed to the air-liquid interface, the microbial film constructed by the three-dimensional model of a microbial film exposed at an air-liquid interface allows microorganisms to grow, proliferate, and express their microbial functions more effectively. Finally, by adjusting a culture time of the microorganisms or an immersion time of the scaffold in the microbial culture medium, a microbial load within the biofilm structure can be precisely controlled, achieving a high degree of controllability over the biofilm.

The present disclosure also provides a preparation method of the above rapidly constructed three-dimensional model of a microbial film exposed at an air-liquid interface, including following steps:
(1) construction of a hydrogel precursor of a core: first, sealing a non-biotoxic and low-temperature congealable hydrogel material and polyvinyl alcohol (PVA) in their dry states, respectively, and then performing high-temperature and high-pressure sterilization at 115° C. to 131° C. for 18-24 min; subsequently, mixing the sterile hydrogel material with the sterile polyvinyl alcohol, and swelling in a mixture in a culture medium required for immobilizing target microorganisms for 15-45 min, dissolving the mixture at 90° C. to 100° C., and maintaining the mixture at 60° C. to 80° C. for later use; dissolving an antimicrobial chitosan hydrochloride in a solution of a non-bio toxic reagent that is soluble or sparingly soluble in water and capable of releasing calcium ions, and performing sterilization by filtering with a 0.22-μm sterile nylon filter head; mixing the prepared hydrogel-polyvinyl alcohol mixed solution with the chitosan hydrochloride solution at a specific mass ratio to ensure that an obtained mixture contains 2-12% of the low-temperature congealable hydrogel, 8-12% of the polyvinyl alcohol, 1-1.5% of the chitosan hydrochloride, 1-1.5% of the calcium ion reagent, and sufficient nutrients, to give a core of a three-dimensional model;
(2) pouring the hydrogel precursor of the core into a silicone mold designed according to a desired structure, sealing the hydrogel precursor of the core at 2° C. to 8° C. for 1-4 h until the low-temperature congealable hydrogel in a liquid state completely solidifies to form a scaffold; allowing the scaffold to undergo freezing at −20° C. to −86° C. for 12-24 h, thawing at room temperature for 12-24 h, and then freezing at −20° C. to −86° C. for additional 12-24 h, and repeating the cycle three times to obtain a scaffold with enhanced gel strength;
(3) construction of a hydrogel precursor for microbial culture: first, sealing sodium alginate in its dry state, and then performing high-temperature and high-pressure sterilization at 115° C. to 131° C. for 18-20 min; subsequently, adding the sterile sodium alginate to a sterile Tris-HCl buffer of 4-6 mmol/L containing salt ions required for immobilizing target microorganisms to finally ensure that an obtained mixture contains 0.6-0.8% of sodium alginate;
(4) culture of immobilized microorganisms: culturing the target microorganisms in a microorganism solution state for 14-18 h, and performing low-temperature centrifugation at 5,000-7,000 rpm at 2° C. to 8° C. for 5-10 min; washing the microorganisms collected by centrifugation with a sterile Tris-HCl buffer of 4-6 mmol/L three times, mixing the microorganisms evenly into the hydrogel precursor for microbial culture, and performing vortex mixing for 1-3 min to form a shell composed of highly biocompatible sodium alginate, which combines with a buffer system and salt ions to ensure normal survival of the microorganisms in a short period of time;
(5) formation of a biofilm-like film: fully immersing the prepared scaffold in the hydrogel precursor for microbial culture containing the immobilized microorganisms, where the chitosan hydrochloride as a cationic polymer first interacts with the sodium alginate as an anionic polymer to form a polyelectrolyte layer which quickly envelopes the entire scaffold core and to create an antimicrobial layer to prevent microbial invasion into the scaffold core; subsequently, gradually releasing the calcium ions in the calcium ion-containing reagent in the scaffold into the hydrogel precursor for microbial culture to gradually form a calcium-alginate hydrogel film around the scaffold core, thus immobilizing the microorganisms in the hydrogel precursor for microbial culture on a surface of the core scaffold, where there is a linear positive correlation between a film thickness and the immersion of the core scaffold within a certain period of time, with the film thickness varying at a rate of 50 μm/min;
(6) stabilization of the model of the biofilm-like film: stabilizing the constructed three-dimensional model of the microorganisms in a sterile Tris-HCl buffer of 4-6 mmol/L containing nutrients required for the immobilized microorganisms at 2° C. to 8° C. for 5-10 min, thus facilitating replenishment of nutrients in the scaffold under action of material exchange, where the chitosan hydrochloride undergoes deprotonated crosslinking and further strengthens the gel strength of the scaffold under action of the Tris-HCl buffer; and
(7) construction of a microbial film exposed at an air-liquid interface: purging the model of the biofilm-like film by high-purity air at a flow rate of 0.4-0.8 L/min for 4-8 min so that the film layer rapidly shrinks to a hydrogel film with a thickness of no more than 50 μm due to a greater dehydration rate of the sodium alginate compared to that of the mixed hydrogel of the scaffold, to compel some microorganisms immobilized therein to be exposed at the air-liquid interface.

Furthermore, the hydrogel material in step (1) is selected from gelatin, agar, agarose, and carrageenan; and the calcium ion reagent is selected from calcium chloride, calcium sulfate, and calcium citrate.

During the construction of the hydrogel precursor for microbial culture in step (3), the microorganisms are uniformly distributed and their amount is controllable. Therefore, during the construction of the in vitro microbial model of biofilm-like film, a microbial load of the model of the biofilm-like film is controlled by adjusting an immersion time of the scaffold.

The three-dimensional model of a microbial film exposed at an air-liquid interface of the present disclosure can be stored at −20° C. for a long term after sterile sealing.

The microbial immobilization state in the three-dimensional model of a microbial film exposed at an air-liquid interface of the present disclosure: the microorganisms immobilized on the in vitro microbial model of the biofilm-like film are in three states:
(1) the unique state is that microorganisms are exposed and survive in the air-liquid interface state by virtue of a three-dimensional porous structure of the calcium alginate so that the microorganisms can be directly exposed to substances in the air, and can also obtain water and nutrients released from the scaffold in this state. However, this state cannot be sustained due to inevitable continuous dehydration from air exposure;
(2) secondly, although a shell shrinks into a film, some immobilized microorganisms still survive in a hydrogel environment similar to a liquid environment, ensuring their long-term survival; and these microorganisms are indirectly exposed to soluble substances in the air by diffusion; and
(3) lastly, some immobilized microorganisms are exposed to or penetrate the polyelectrolyte layer due to proliferation or pressure caused by the shell shrinkage so that the microorganisms die immediately.

The polyvinyl alcohol can be replaced with any non-biotoxic material that can form a hydrogel through a non-toxic crosslinking process.

The present disclosure also provides a hydrogel bead containing microorganisms immobilized at an air-liquid interface in an exposed state and a preparation method thereof. The hydrogel bead enables the rapid immobilization of any culturable microorganisms to form a biofilm-like state exposed at the air-liquid interface, which can be used for in-situ, rapid, and multi-toxicity endpoint gaseous exposures of microorganism-based air pollutants.

The hydrogel bead containing microorganisms immobilized at the air-liquid interface in an exposed state of the present disclosure has a multi-layered core-shell structure, with a core configured to be a hydrogel material containing gradually releasable nutrients, a shell configured to be a highly biocompatible hydrogel film enabling microorganisms to survive and proliferate thereon. After immersion in nutrient solution and purging with air for some time, the shell shrinks to a layer of film due to differences in swelling and dehydration rates between the core hydrogel material and the shell hydrogel material, to compel microorganisms inhabit a unique air-liquid interface. An extremely thin antimicrobial polyelectrolyte layer (i.e., bilayer) is between the core and the shell of the bead, which allows an exchange of substances, including nutrients between the core and the shell while preventing the microorganisms from migrating to a more nutritive core. This ensures a uniform distribution of microorganisms on a surface of the hydrogel bead and maintains their continuous exposure at the air-liquid interface. An outer surface of the shell of the hydrogel bead is directly exposed to polluted air, while an inner surface receives nutrients released from the core, which allows microorganisms to be exposed to polluted air while enabling normal survival and proliferation at the air-liquid interface.

In addition, in the present disclosure, a volume of the core and a volume of the shell satisfy the following relationship:

$$\frac{{v}_{s p h e r e}}{{v}_{s h e l l}} = \frac{{d}^{3}}{3 {d}^{2} h - 3 d {h}^{2} + {h}^{3}}$$

where d is a diameter of the hydrogel bead, and h is a thickness of the layer before shrinkage.

Taking a hydrogel bead with a diameter of 5 mm as an example, the thickness of the layer before shrinkage is 0.2 mm, and the volume of the core is 8 times that of the shell. Compared to a microorganism solution state, the microorganisms on the surface of the hydrogel bead can access nutrients equivalent to 8 times their own volume. Aerobic microorganisms can obtain a larger amount of oxygen at the air-liquid interface. Therefore, when exposed to the air-liquid interface, microorganisms immobilized in the hydrogel bead can grow, proliferate, and maintain their microbial functions more effectively.

The hydrogel bead containing microorganisms immobilized at the air-liquid interface in an exposed state has a multi-layered structure. The multi-layered structure is formed by one-step self-assembly without the need for complex manual operations.

The present disclosure also provides a preparation method of the hydrogel bead, including the following steps:
(1) construction of a hydrogel precursor of a core: first, sealing a non-biotoxic and low-temperature congealable hydrogel material in its dry state, and then performing high-temperature and high-pressure sterilization at 120° C. to 131° C. for 18-24 min; subsequently, swelling the sterile hydrogel material in a culture medium required for immobilizing target microorganisms for 15-45 min, dissolving the hydrogel material at 55° C. to 80° C., and maintaining the hydrogel material in its sealing state at 50° C. to 70° C. for later use; dissolving chitosan hydrochloride in a non-biotoxic reagent solution that is soluble or sparingly soluble in water and capable of releasing calcium ions, and performing sterilization by filtering with a 0.22 μm sterile nylon filter head; mixing the prepared hydrogel solution with the chitosan hydrochloride solution in a mass ratio to finally ensure that an obtained mixture contains 8-12% of the hydrogel material, 1-1.5% of the chitosan hydrochloride, 1-1.5% of the calcium ion reagent, and sufficient nutrients;
(2) construction of a hydrogel precursor of a shell: first, sealing sodium alginate in its dry state, and then performing high-temperature and high-pressure sterilization at 120° C. to 131° C. for 18-22 min; subsequently, adding a certain amount of the sterile sodium alginate to a sterile Tris-HCl buffer of 4-6 mmol/L containing salt ions required for immobilizing target microorganisms to finally ensure that an obtained mixture contains 0.6-0.8% of sodium alginate;
(3) culture of immobilized microorganisms: culturing the target microorganisms in a microorganism solution state for 14-18 h, and performing low-temperature centrifugation at 5,000-7,000 rpm at 2° C. to 8° C. for 5-10 min; washing the microorganisms collected by centrifugation with a sterile Tris-HCl buffer of 4-6 mmol/L three times, and mixing the microorganisms uniformly into the hydrogel precursor of the shell and performing vortex mixing for 1-3 min to form a shell composed of highly biocompatible sodium alginate, which combines with an appropriate buffer system and salt ions, to ensure normal survival of the microorganisms in a short period;
(4) self-assembly of the hydrogel bead: suctioning the hydrogel precursor of the core maintained at 50° C. to 70° C. by a peristaltic pump at a rate of 1-2 ml/min, and keeping a pipeline of the peristaltic pump in an attemperator set at 50° C. to 70° C. throughout the process to prevent the hydrogel precursor of the core from premature solidification due to; slowly and evenly adding the suctioned hydrogel precursor of the core dropwise to the hydrogel precursor of the shell containing the immobilized microorganisms from a height of 10-20 cm above a liquid surface of the hydrogel precursor of the shell. The chitosan hydrochloride as a cationic polymer interacts with the sodium alginate as an anionic polymer to form a polyelectrolyte layer which quickly envelopes the hydrogel precursor of the core in a liquid state and creates a liquid sphere structure to prevent the escape of the remaining gel components; subsequently, Ca²⁺ in the hydrogel precursor of the core is gradually releasing into the hydrogel precursor of the shell, to gradually form a calcium-alginate hydrogel shell around the liquid sphere, thus immobilizing the microorganisms in the hydrogel precursor of the shell. There is a strong linear positive correlation between a shell thickness and self-assembly time within a certain period, with the shell thickness varying at a rate of 50 μm/min; and producing the hydrogel beads with different initial particle thicknesses by controlling the self-assembly time;
(5) stabilization of the hydrogel bead: stabilizing the self-assembled hydrogel bead in a sterile Tris-HCl buffer of 4-6 mmol/L containing nutrients required for the immobilized microorganisms at 2° C. to 8° C. for 5-15 min, thus facilitating replenishment of nutrients in the core under action of material exchange, and promoting cooling and solidification of gelatin in the core in the low-temperature environment to form a solid hydrogel core, where the chitosan hydrochloride in the core undergoes deprotonated crosslinking to form an interpenetrating polymer network structure with the gelatin under action of the Tris-HCl buffer; and
(6) shrinkage of the shell of the hydrogel bead: completing initial shrinkage of the shell of the hydrogel bead with the core swelling due to a higher swelling rate of the gelatin compared to that of the calcium alginate during the stabilization; and purging the stabilized hydrogel bead by high-purity air at a flow rate of 0.4-0.8 L/min for 4-8 min so that the shell quickly shrinks to a hydrogel film with a thickness of no more than 50 μm due to a greater dehydration rate of the calcium alginate compared to that of the gelatin to compel some microorganisms immobilized therein to be exposed at the air-liquid interface.

Furthermore, the hydrogel material in step (1) includes but is not limited to gelatin, agar, agarose, and carrageenan, preferably gelatin; and the calcium ion reagent includes but is not limited to calcium chloride, calcium sulfate, and calcium citrate, preferably calcium chloride.

During the self-assembly of the hydrogel bead to prepare the hydrogel precursor of the shell containing the immobilized microorganisms in step (4), the microorganisms are uniformly distributed and their amount is controllable. Therefore, during the self-assembly, an amount of the microorganisms immobilized on the hydrogel bead is controlled by adjusting the self-assembly time of the hydrogel bead.

Storage of the hydrogel bead: the self-assembled hydrogel bead is immersed in a nutrient solution containing 40% of glycerol for 20-40 min and stored at −20° C.; the cryopreserved hydrogel bead is then stabilized in a sterile Tris-HCl buffer of 4-6 mmol/L containing nutrients required for the immobilized microorganisms for 20-40 min, and then incubated in a constant-temperature incubator for 2-3 h; and the hydrogel bead is purged by high-purity air at a flow rate of 0.5 L/min for 5 min to re-shrink the shell.

The microorganisms immobilized on the hydrogel bead of the present disclosure are in three states:
(1) the unique state is that the microorganisms are exposed and survive in the air-liquid interface state by virtue of a three-dimensional porous structure of the calcium alginate so that the microorganisms can be directly exposed to substances in the air, and can also obtain water and nutrients released from the core in this state. However, this state cannot be sustained due to inevitable continuous dehydration from air exposure.
(2) Although the shell shrinks into a film, some immobilized microorganisms still survive in a hydrogel environment similar to a liquid environment, ensuring their long-term survival. These microorganisms are indirectly exposed to soluble substances in the air by diffusion; and
(3) some immobilized microorganisms are exposed to or penetrate the polyelectrolyte layer due to proliferation or pressure caused by shell shrinkage so that the microorganisms die immediately.

The present disclosure has the following beneficial effects:

The present disclosure provides a rapidly constructed three-dimensional model of a microbial film exposed at an air-liquid interface, which circumvents the prolonged natural growth cycle of microbial films and overcomes the limitation that certain microorganisms are unable to form microbial films naturally. Within a few minutes (e.g., 1-10 min), any culturable microorganisms can be rapidly immobilized to form a biofilm-like state in any three-dimensional structure, while sustaining normal microbial growth, proliferation, and functions. The model breaks through the limitation that microbial films can only be maintained in a culture medium and allows their exposure to air, thereby enabling the research of microbial behaviors under air exposure. The in vitro microbial model of biofilm-like film exposed at the air-liquid interface compels some microorganisms to be exposed in a unique air-liquid interface state. The three-dimensional scaffold can uniformly and controllably deliver nutrients and water to the immobilized microorganisms' biofilm-like film, enhancing the nutrient utilization efficiency of the immobilized microorganisms, to ensure that the microorganisms can continue to grow, proliferate, and maintain their biological functions even when partially exposed to air. Chitosan hydrochloride, a potent antimicrobial agent, is unable to be released into the biofilm-like structure to harm the immobilized microorganisms; instead, it serves as a barrier to prevent microorganisms from entering the interior of the more nutritive scaffold. This guarantees that the model of the biofilm-like film remains continuously and stably exposed at the air-liquid interface. The entire model of the biofilm-like film can be constructed into any shape and structure as required, and the combination of immobilizing microorganisms is not limited by the immobilization process, making the whole method highly scalable, well-stabilized and reproducible. Furthermore, due to the stable physical barrier provided by the hydrogel of the biofilm-like film and the consistent nutrient and water supply from the three-dimensional scaffold, the microorganisms immobilized on model exhibit stronger anti-interference capabilities compared to those on naturally grown microbial films. In the future, the three-dimensional model of a microbial film exposed at an air-liquid interface can provide support for research on microbial behaviors, microbial interactions, drug effects, gaseous drug administration, sterilization, and microbial air exposure behaviors, and it will significantly reduce the costs and enhance the efficiency of related research.

The present disclosure also provides a hydrogel bead containing microorganisms immobilized at an air-liquid interface in an exposed state, which has a multi-layered core-shell structure with a surface of a hydrogel film containing microorganisms immobilized at the air-liquid interface in an exposed state. The core of the hydrogel bead can uniformly and controllably deliver nutrients and water to the shell immobilizing microorganisms, enhancing the nutrient utilization efficiency of the immobilized microorganisms, to ensure that the microorganisms can continue to grow, proliferate, and maintain their biological functions even when partially exposed to the air. One antimicrobial polyelectrolyte bilayer exists between the core and the shell, which prevents microorganisms from entering a more nutritive core and guarantees that the immobilized microorganisms remain continuously and stably exposed at the air-liquid interface. In addition, the constructed hydrogel bead exhibits ultra-high transparency similar to that of pure water, enabling the detection of fluorescent signals expressed by the immobilized microorganisms without interference. Together with a stable and controllable microbial load, the hydrogel bead containing immobilized microorganisms demonstrates good consistency and stability, with an average error in fluorescent signals of 4.34%. Due to the stable physical barrier provided by the shell hydrogel and the consistent nutrient supply from the core, the microorganisms immobilized on the hydrogel bead exhibit stronger anti-interference capabilities compared to those in a microorganism solution state. In combination with specialized storage procedures, the microorganisms immobilized in the hydrogel bead can be stably stored for over a month while still maintaining their biological functions. In the future, by integrating atmospheric preconcentration equipment and online fluorescence detection devices with the hydrogel-bead-based exposure system, it will be possible to achieve low-cost, rapid, and customized assessments of toxic effects in real air environments.

### BRIEF DESCRIPTION OF THE DRAWINGS

FIG. 1 shows a structural diagram of a three-dimensional model of a microbial film exposed at an air-liquid interface of the present disclosure (in a circle). Numerals in FIG. 1: 5: microbial biofilm-like film; 6: microorganisms immobilized on the biofilm-like film; 7: antimicrobial polyelectrolyte layer; and 8: scaffold (in a circle).

FIG. 2 shows macro images of models of microbial films exposed at a liquid interface with three different three-dimensional structures (film-like, round wafer-like, and columnar).

FIG. 3 shows a diagram indicating a comparison between the fluorescence intensity of 29 recombinant Escherichia coli strains carrying plasmids with different genetic pathways after immobilization using a model of a biofilm-like film and that of a microorganism solution before immobilization.

FIG. 4 shows staining images of apoptosis and necrosis of microorganisms after immobilizing competent Escherichia coli strains on a model of a biofilm-like film and exposing them to air for a period, where (a) represents the un-immobilized competent Escherichia coli strains, and (b) represents the competent Escherichia coli strains on the model of the biofilm-like film.

FIG. 5 shows a macro image of a hydrogel bead containing microorganisms immobilized at an air-liquid interface in an exposed state. Numerals in FIG. 5: 1: interpenetrating polymer hydrogel core of gelatin and chitosan hydrochloride; 2: sodium alginate-chitosan hydrochloride polyelectrolyte layer; and 3: calcium-alginate hydrogel shell.

FIG. 6 shows a microscopic observation diagram of a hydrogel bead containing microorganisms immobilized at an air-liquid interface in an exposed state.

FIG. 7 shows a microscopic observation diagram of the shrinkage process of a shell of a hydrogel bead containing microorganisms immobilized at an air-liquid interface in an exposed state.

FIG. 8 shows a microscopic observation diagram of the growth and proliferation of microorganisms in a shell within 24 h after Escherichia coli strains were immobilized on a hydrogel bead containing microorganisms immobilized at an air-liquid interface in an exposed state.

FIG. 9 shows a comparison photo of the transparency of a calcium-alginate bead and a hydrogel bead, as well as a comparison diagram of absorbance spectra of the hydrogel bead, pure water and air.

FIG. 10 shows a comparison diagram of fluorescent signals of unimmobilized recombinant Escherichia coli strains and recombinant Escherichia coli strains immobilized by a hydrogel bead under a fluorescence microscope.

FIG. 11 shows a diagram indicating the response stability of a hydrogel bead containing immobilized recombinant Escherichia coli strains towards the genotoxicity of gaseous pollutants over 30 days of storage.

FIG. 12 shows a genotoxicity heatmap of in-situ measurements of gaseous methyl methane sulfonate using a hydrogel bead with 29 immobilized recombinant Escherichia coli strains carrying plasmids with different genetic pathways.

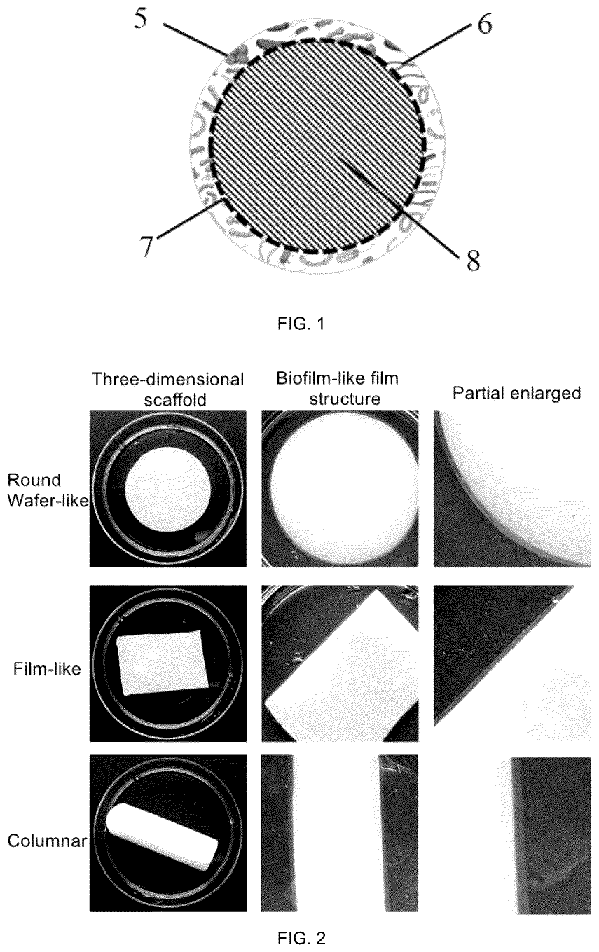

### DETAILED DESCRIPTION

The present disclosure will be further described below with reference to the accompanying drawings.

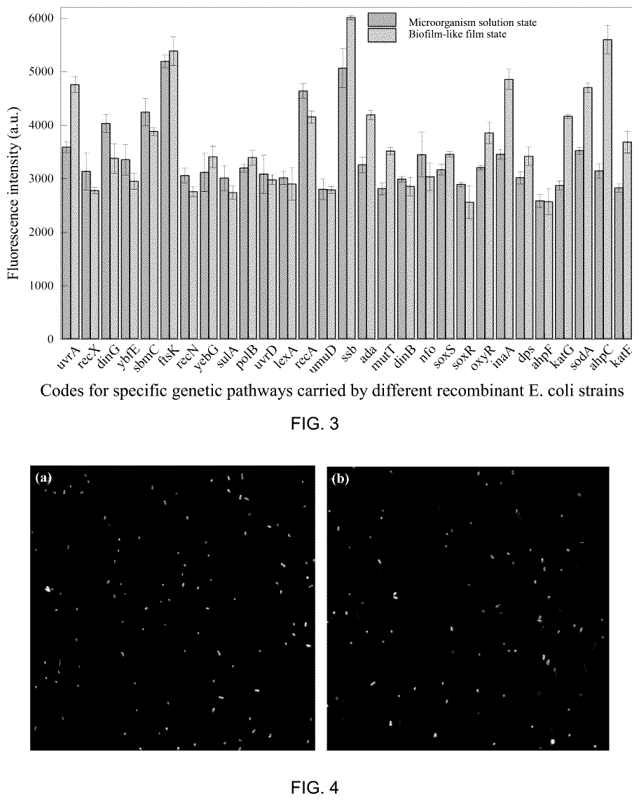

Embodiment 1: Preparation of a three-dimensional model of a microbial film exposed at an air-liquid interface.
(1) construction of a hydrogel precursor of a core: first, agarose and polyvinyl alcohol were sealed in their dry states, respectively, and then high-temperature and high-pressure sterilization was performed at 121° C. for 20 min; subsequently, the sterile agarose was mixed with the sterile polyvinyl alcohol, and the mixture was made to swell in a culture medium required for immobilizing target microorganisms for 30 min, the mixture was dissolved at 95° C., and maintained at 80° C. for later use; a certain amount of antimicrobial chitosan hydrochloride was dissolved in a solution of calcium chloride, and sterilization was performed by filtering with a 0.22 μm sterile nylon filter head; the prepared agarose-polyvinyl alcohol mixed solution was mixed with the chitosan hydrochloride solution in a mass ratio of 1:1 to ensure that an obtained mixture contains 2.5% of the agarose, 8% of the polyvinyl alcohol, 1% of the chitosan hydrochloride, 1.5% of the calcium chloride, and sufficient nutrients;
(2) the hydrogel precursor of the core was poured into silicone molds with round wafer-like, film-like, and columnar structures required for the present embodiment, and sealed at 4° C. for 1 h until the liquid agarose completely solidified to form three scaffolds as required; the scaffolds were allowed to undergo freezing at −20° C. for 24 h, thawing at room temperature for 12 h, and then freezing at −20° C. for additional 24 h, and the cycle was repeated three times to obtain scaffolds with enhanced gel strength;
(3) construction of a hydrogel precursor for microbial culture: first, sodium alginate was sealed in its dry state, and then high-temperature and high-pressure sterilization was performed at 121° C. for 20 min; subsequently, a certain amount of the sterile sodium alginate was added to a sterile Tris-HCl buffer of 5 mmol/L containing salt ions required for immobilizing target microorganisms to finally ensure that an obtained mixture contains 0.8% of the sodium alginate;
(4) culture of immobilized microorganisms: in this Embodiment, 29 recombinant Escherichia coli strains carrying plasmids with different genetic pathways were taken as immobilization targets; the aforesaid microorganisms were cultured in a microorganism solution state for 14-18 h, and low-temperature centrifugation was performed at 5,000 rpm at 4° C. for 10 min; the microorganisms collected by centrifugation were washed with a sterile Tris-HCl buffer of 5 mmol/L three times, and the microorganisms were mixed uniformly into the hydrogel precursor for microbial culture, and vortex mixing was performed for 3 min to form a shell composed of highly biocompatible sodium alginate, which combines with an appropriate buffer system and salt ions to ensure normal survival of the microorganisms in a short period of time;
(5) formation of biofilm-like films: the prepared scaffolds were fully immersed in the hydrogel precursors for microbial culture containing different recombinant Escherichia coli strains, where the chitosan hydrochloride as a cationic polymer first interacted with the sodium alginate as an anionic polymer to form polyelectrolyte layers which quickly enveloped the entire core scaffolds to create antimicrobial layers to prevent microbial invasion into the core scaffolds; subsequently, the calcium ions in the calcium chloride in the scaffolds were gradually released into the hydrogel precursors for microbial culture to gradually form calcium-alginate hydrogel films around the core scaffolds, thus immobilizing the recombinant Escherichia coli strains in the hydrogel precursors for microbial culture on surfaces of the core scaffolds, where there was a linear positive correlation between a film thickness and the immersion of the core scaffolds, with the film thickness varying at a rate of 50 μm/min;
(6) stabilization of the models of the biofilm-like films: during the construction of the three-dimensional models of the biofilm-like films of recombinant Escherichia coli strains, the loss of nutrients was inevitable; therefore, the constructed three-dimensional models of the biofilm-like films were stabilized in a sterile Tris-HCl buffer of 5 mmol/L containing nutrients required for the recombinant Escherichia coli strains at 4° C. for 10 min, thus facilitating replenishment of nutrients in the core scaffold under action of material exchange, where the chitosan hydrochloride in the core underwent deprotonated crosslinking and further strengthened the gel strength of the scaffolds under action of the Tris-HCl buffer; and
(7) construction of microbial films exposed at an air-liquid interface: the study of the expression of specific genes in the recombinant Escherichia coli strains in an air-exposed state was required in this Embodiment; therefore, the models of the biofilm-like films were purged by high-purity air at a flow rate of 0.5 L/min for 5 min so that the film layers rapidly shrank to hydrogel films with a thickness of no more than 50 μm due to a greater dehydration rate of the sodium alginate as compared to that of the mixed hydrogel of the three-dimensional nutrient scaffolds, to compel some microorganisms immobilized therein to be exposed at the air-liquid interface.
(8) In this Embodiment, three-dimensional models of microbial films exposed at an air-liquid interface were prepared in three shapes: film-like, round wafer-like, and columnar. 29 recombinant Escherichia coli strains carrying plasmids with different genetic pathways were rapidly immobilized onto these three-dimensional models. The immobilization duration was 200 s, resulting in biofilm-like film structures with a thickness of approximately 0.2 mm. Macro photography clearly represented the presence of the biofilm-like film structures. Regardless of the shape of the three-dimensional scaffold, a transparent layer of biofilm-like film structure containing microorganisms could be rapidly formed on its surface. Moreover, the entire model exhibited remarkable mechanical strength and stability, with a uniform thickness of the biofilm-like film structure across all parts of the scaffold. See FIG. 2 for details.
(9) Following the aforementioned steps, 29 film-like, 29 round wafer-like, and 29 columnar biofilm-like film structures were constructed and placed in a constant-temperature incubator at 37° C. and humidity of 60% for incubation for 2 h. Subsequently, they were sequentially positioned in a live-cell imaging system to detect the fluorescent signal intensity on their surfaces, which represented the expression levels of specific genetic pathways. The surfaces of all models of biofilm-like films exhibited a certain intensity of fluorescent signals emitted by the recombinant microorganisms, with fluorescence intensities ranging from 2,556 a.u. to 6,013 a.u. The gene expression levels were slightly higher than those of the recombinant Escherichia coli strains in an antimicrobial solution state before immobilization. See FIG. 3 for details.

Embodiment 2: Survival state of the microorganisms immobilized on the three-dimensional model of a microbial film exposed at an air-liquid interface (the specific steps are the same as in Embodiment 1 if not described).
(1) In this embodiment, the core scaffold of the three-dimensional model was designed as a bead with a diameter of 6 mm, with competent Escherichia coli DH5a as the immobilization target. The immobilization duration was 200 s, resulting in a biofilm-like film structure with a thickness of approximately 0.2 mm;
(2) the constructed model was purged by pure air at a flow rate of 1 L/min for 30 min; following this, the biofilm-like film structure was peeled off from its scaffold, and the immobilized competent Escherichia coli were liquefied and recovered through a mixture of 0.055 mol/L citric acid and 0.005 mol/L phosphate-buffered saline (PBS); and
(3) the recovered Escherichia coli were stained using a fluorescence kit for apoptosis and necrosis, and competent Escherichia coli without immobilization was taken as a control. As shown in FIG. 4, after immobilization by the three-dimensional model of a microbial film exposed at an air-liquid interface and exposure to air for 30 min, no dead cells were found, with only a small portion of cells entering early apoptosis, while most of the immobilized microorganisms remained viable. The result indicates that the model of the biofilm-like film could make the microorganisms maintain normal growth, proliferation, and microbial functions while exposed to a gaseous environment.

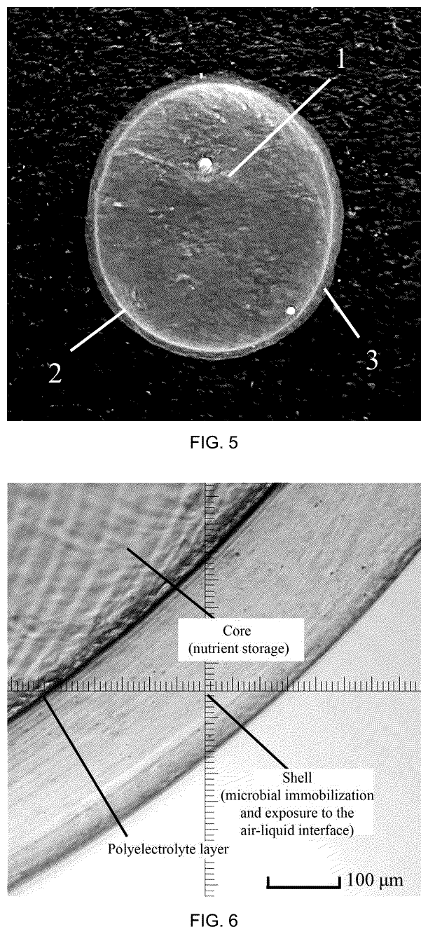

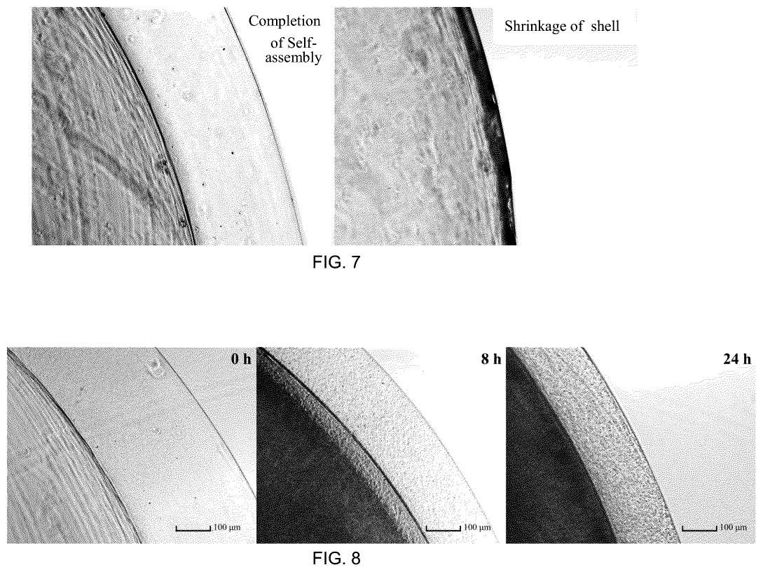

Embodiment 3: Preparation of a hydrogel bead containing microorganisms immobilized at an air-liquid interface in an exposed state.
(1) construction of a hydrogel precursor of the core: first, gelatin was sealed in its dry state, and then high-temperature and high-pressure sterilization was performed at 121° C. for 20 min; subsequently, the sterile gelatin was made to swell in a culture medium required for immobilizing target microorganisms for 30 min, dissolved at 55° C., and maintained in its sealing state at 50° C. for later use; a certain amount of chitosan hydrochloride was dissolved in a calcium chloride solution, and sterilization was performed by filtering with a 0.22-μm sterile nylon filter head; the prepared gelatin solution was mixed with the chitosan hydrochloride solution in a mass ratio of 1:1 to finally ensure that an obtained mixture contains 10% of the gelatin, 1% of the chitosan hydrochloride, 1.5% of the calcium chloride, and sufficient nutrients;
(2) construction of a hydrogel precursor of the shell: first, sodium alginate was sealed in its dry state, and then high-temperature and high-pressure sterilization was performed at 121° C. for 20 min; subsequently, the sterile sodium alginate was added to sterile Tris-HCl buffer of 5 mmol/L containing salt ions required for immobilizing target microorganisms to finally ensure that an obtained mixture contains 0.6% of the sodium alginate;
(3) culture of immobilized microorganisms: the target microorganisms were cultured in a microorganism solution state for 14-18 h, and low-temperature centrifugation was performed at 5,000 rpm at 4° C. for 10 min; the microorganisms collected by centrifugation were washed with a sterile Tris-HCl buffer of 5 mmol/L three times, and the microorganisms were mixed evenly into the hydrogel precursor of the shell, and vortex mixing was performed for 3 min to form a shell composed of highly biocompatible sodium alginate, which combines with an appropriate buffer system and salt ions to ensure normal survival of the microorganisms in a short period of time;
(4) self-assembly of the hydrogel beads: the hydrogel precursor of the core maintained at 50° C. was suctioned by a peristaltic pump at a rate of 1.5 ml/min, and a pipeline of the peristaltic pump was kept in an attemperator set at 50° C. throughout the process to prevent the hydrogel precursor of the core from premature solidification due to cooling; the suctioned hydrogel precursor of the core was slowly and evenly dripped into the hydrogel precursor of the shell containing the immobilized microorganisms from a height of 15 cm above a liquid surface of the hydrogel precursor of the shell, where the chitosan hydrochloride as a cationic polymer interacted with the sodium alginate as an anionic polymer to form a polyelectrolyte layer that quickly enveloped the hydrogel precursor of the core in a liquid state and to create a liquid sphere structure to prevent the escape of remaining gel components; subsequently, Ca²⁺ in the hydrogel precursor of the core was gradually released into the hydrogel precursor of the shell to gradually form a calcium-alginate hydrogel shell around the liquid spheres, thus immobilizing the microorganisms in the hydrogel precursor of the shell, where there was a strong linear positive correlation between a shell thickness and an self-assembly time within a certain period of time, with the shell thickness varying at a rate of 50 μm/min. The self-assembly time was set to 200 s in this Embodiment, and the initial shell thicknesses of the produced hydrogel beads were about 0.2 mm (as shown in FIGS. 5-6).
(5) stabilization of the hydrogel beads: during the self-assembly, the loss of nutrients was inevitable; therefore, the self-assembled hydrogel beads were stabilized in a sterile Tris-HCl buffer of 5 mmol/L containing nutrients required for the immobilized microorganisms at 4° C. for 10 min, thus facilitating replenishment of nutrients in the core under action of material exchange, and promoting cooling and solidification of gelatin in the core in the low-temperature environment to form a solid hydrogel core was promoted, where the chitosan hydrochloride in the core underwent deprotonated crosslinking to form an interpenetrating polymer network structure with the gelatin under action of the Tris-HCl buffer; and
(6) shrinkage of the shell of the hydrogel beads: initial shrinkage of the shell layers of the hydrogel beads was completed with the core swelling due to a higher swelling rate of the gelatin compared to that of the calcium alginate during the stabilization; the stabilized hydrogel beads were purged by high-purity air at a flow rate of 0.5 L/min for 5 min so that the shell quickly shrank to a hydrogel film with a thickness of no more than 50 μm due to a greater dehydration rate of the calcium alginate compared to that of the gelatin to compel some microorganisms immobilized therein to be exposed at the air-liquid interface (as shown in FIG. 7).
(7) The completed immobilized hydrogel beads were placed in a constant-temperature incubator at 37° C. and humidity of 60%. Subsequently, they were suctioned and immersed in PBS at 0 h, 8 h, and 24 h for observation under a microscope, and the rapid growth and proliferation of the microorganisms in the shells of the hydrogel beads could be clearly observed (as shown in FIG. 8).

During the self-assembly of the hydrogel beads to prepare the hydrogel precursor of the shell containing the immobilized microorganisms in step (4), the amount of the microorganisms immobilized on the hydrogel beads was controlled by adjusting the amount of microorganisms added to the hydrogel precursor of the shell and the self-assembly time of the hydrogel beads.

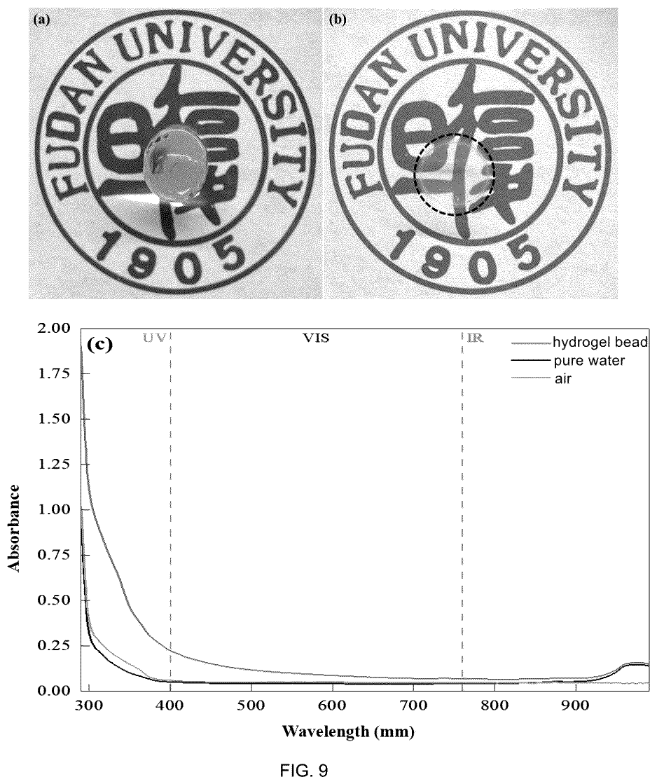

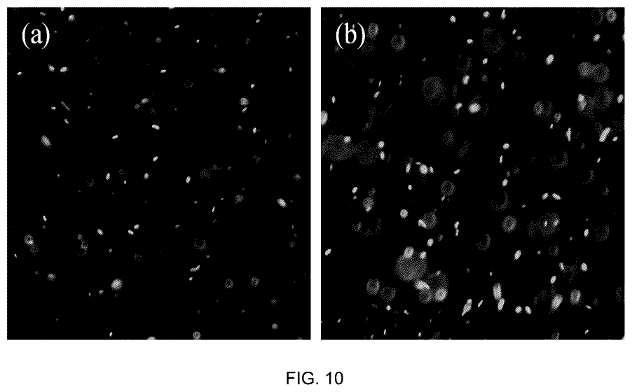

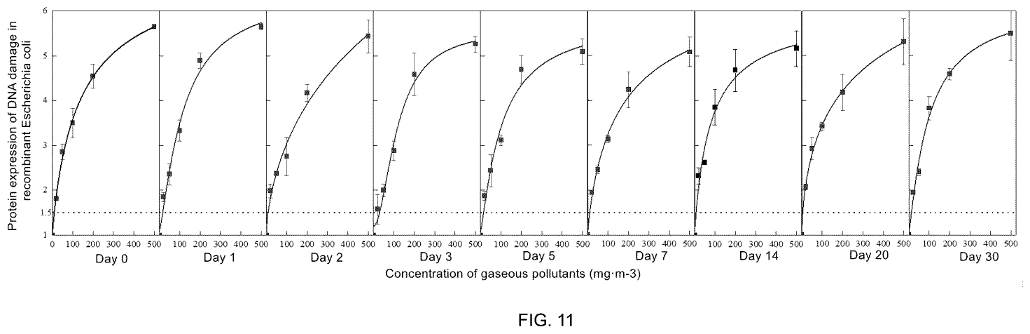

Embodiment 4: Use of the hydrogel beads for the immobilization of recombinant Escherichia coli strains carrying plasmids with special genetic pathways for the detection of genotoxicity of gaseous pollutants:
(1) Compared to conventional calcium-alginate hydrogel beads, the constructed hydrogel beads exhibited higher transparency. Its absorption spectra within the full spectral wavelength range were similar to that of pure water, demonstrating that the transparency of the constructed hydrogel beads was comparable to pure water, and the recombinant Escherichia coli strains carrying plasmids with special genetic pathways could express their fluorescent signals without interference (as shown in FIG. 9);
(2) Recombinant Escherichia coli strains with the rec A genetic pathway were immobilized within the hydrogel beads and exposed to a 50 mg/L solution of dimethyl sulfate. Compared to the recombinant Escherichia coli strains that were not immobilized within the hydrogel beads, those immobilized exhibited stronger fluorescent signals when exposed to the same concentration of dimethyl sulfate (as shown in FIG. 10); and
(3) The self-assembled hydrogel beads containing immobilized recombinant Escherichia coli strains with the rec A genetic pathway were immersed in a nutrient solution containing 40% glycerol for 30 min and then stored at −20° C. At intervals of 0, 1, 2, 3, 5, 7, 14, 20, and 30 days, the cryopreserved hydrogel beads were suctioned and placed in a sterile Tris-HCl buffer of 5 mmol/L containing the nutrients required for the immobilized recombinant Escherichia coli strains for stabilization for 30 min before being incubated in a constant-temperature incubator for 2-3 h. The hydrogel beads were then suctioned and purged by high-purity air at a flow rate of 0.5 L/min for 5 min to re-shrink the shell. Subsequently, they were exposed to different concentrations of gaseous dimethyl sulfate-contaminated air. The result indicates that, even after long-term storage at low temperatures, the hydrogel beads containing immobilized recombinant Escherichia coli strains maintained stable sensitivity and linearity in response to pollutants (as shown in FIG. 11).

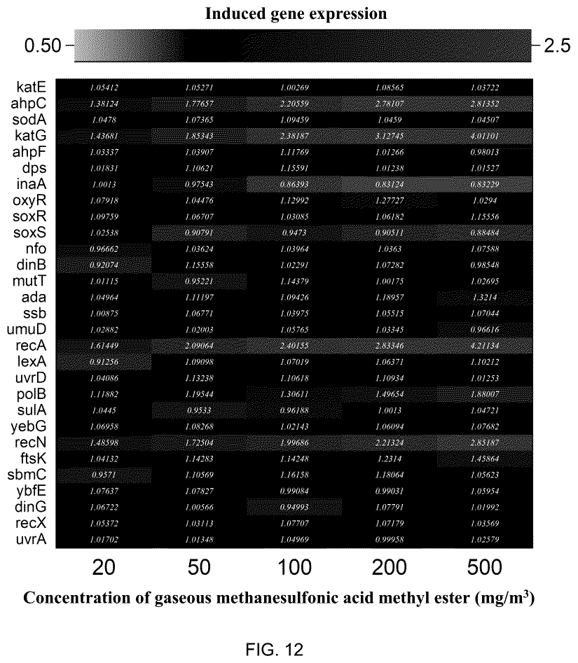

Embodiment 5: Immobilization of 29 recombinant Escherichia coli strains carrying plasmids with different genetic pathways by hydrogel beads for genotoxicity detection of pollutants, including DNA damage, DNA repair, and oxidative stress:
(1) The nutrient in the core was a modified M9 medium, including MgCl₂, CaCl₂), NaCl, NH₄Cl, Tris-HCl (pH-7.4) buffer, and glucose, to ensure the continued survival of the recombinant Escherichia coli strains when exposed at the air-liquid interface.
(2) The hydrogel beads containing immobilized 29 recombinant Escherichia coli strains, were assembled in a specially designed gaseous exposure system. Different concentrations (ranging from 20 to 500 mg/m³) of gaseous methanesulfonic acid methyl ester were introduced into the exposure system for simultaneous exposure. This setup successfully enabled the generation of a genotoxicity heat map under in-situ exposure to gaseous methanesulfonic acid methyl ester; and
(3) As the exposure concentration increased, significant upregulation of the ahp C and Kat G genes indicated that gaseous methanesulfonic acid methyl ester induced pronounced oxidative stress effects based on alkyl peroxides and hydroxyl radicals. Additionally, the significant upregulation of the rec A, pol B, and rec N gene pathways suggested that gaseous methanesulfonic acid methyl ester caused severe DNA damage in the tested microorganisms, prompting them to initiate DNA repair and recombination processes. Conversely, downregulation of the Ina A and sox R genes gaseous methanesulfonic acid methyl ester indicated that inhibited the SOS response of the tested microorganisms. In summary, a rapid analysis of the genotoxicity fingerprint of gaseous methanesulfonic acid methyl ester was conducted from the perspective of in-situ exposure. See FIG. 12 for details.

1. A preparation method of a rapidly constructed three-dimensional model of a microbial film exposed at an air-liquid interface, comprising:
(1) construction of a hydrogel precursor of a core: first, sealing a non-biotoxic and low-temperature congealable hydrogel material and polyvinyl alcohol in their dry states, respectively, and then performing high-temperature and high-pressure sterilization at 115° C. to 131° C. for 18-24 min; subsequently, mixing the sterile hydrogel material with the sterile polyvinyl alcohol and swelling the mixture in a culture medium required for immobilizing target microorganisms for 15-45 min, dissolving the mixture at 90° C. to 100° C., and maintaining the mixture at 60° C. to 80° C. for later use; dissolving an antimicrobial chitosan hydrochloride in a solution of a non-biotoxic reagent that is soluble or sparingly soluble in water and capable of releasing calcium ions, and performing a sterilization by filtering with a 0.22 μm sterile nylon filter head; mixing the prepared hydrogel-polyvinyl alcohol mixed solution with the chitosan hydrochloride solution in a specific mass ratio to ensure that an obtained mixture contains 2-12% of the low-temperature congealable hydrogel, 8-12% of the polyvinyl alcohol, 1-1.5% of the chitosan hydrochloride, 1-1.5% of the calcium ion reagent, and sufficient nutrients; to give a core of a three-dimensional model;
(2) pouring the hydrogel precursor of the core into a silicone mold designed according to a desired structure, sealing the hydrogel precursor of the core at 2° C. to 8° C. for 1-4 h until the low-temperature congealable hydrogel in a liquid state completely solidifies to form a scaffold; allowing the scaffold to undergo freezing at −20° C. to −86° C. for 12-24 h, thawing at room temperature for 12-24 h, and then freezing at −20° C. to −86° C. for additional 12-24 h, and repeating the cycle three times to obtain a scaffold with enhanced gel strength;
(3) construction of a hydrogel precursor for microbial culture: first, sealing sodium alginate in its dry state, and then performing high-temperature and high-pressure sterilization at 115° C. to 131° C. for 18-20 min; subsequently, adding the sterile sodium alginate to a sterile Tris-HCl buffer of 4-6 mmol/L containing salt ions required for immobilizing target microorganisms to finally ensure that an obtained mixture contains 0.6-0.8% of the sodium alginate;
(4) culture of immobilized microorganisms: culturing the target microorganisms in a microorganism solution state for 14-18 h, and performing low-temperature centrifugation at 5,000-7,000 rpm at 2° C. to 8° C. for 5-10 min; washing the microorganisms collected by centrifugation with a sterile Tris-HCl buffer of 4-6 mmol/L three times, mixing the microorganisms evenly into the hydrogel precursor for microbial culture, and performing a vortex mixing for 1-3 min to form a shell composed of highly biocompatible sodium alginate, which combines with a buffer system and salt ions to ensure normal survival of the microorganisms in a short period;
(5) formation of a biofilm-like film: fully immersing the prepared scaffold in the hydrogel precursor for microbial culture containing the immobilized microorganisms, wherein the chitosan hydrochloride as a cationic polymer first interacts with the sodium alginate as an anionic polymer to form a polyelectrolyte layer which quickly envelopes the entire core scaffold to create an antimicrobial layer to prevent microbial invasion into the core scaffold; subsequently, gradually releasing the calcium ions in the calcium ion-containing reagent in the scaffold into the hydrogel precursor for microbial culture to gradually form a calcium alginate hydrogel film around the core scaffold, thus immobilizing the microorganisms in the hydrogel precursor for microbial culture on a surface of the core scaffold, wherein there is a linear positive correlation between a film thickness and the immersion of the core scaffold within a certain period of time, with the film thickness varying at a rate of 50 μm/min;
(6) stabilization of the model of the biofilm-like film: stabilizing the constructed three-dimensional model of the microorganisms in a sterile Tris-HCl buffer of 4-6 mmol/L containing nutrients required for the immobilized microorganisms at 2° C. to 8° C. for 5-10 min, thus facilitating replenishment of nutrients in the scaffold under action of material exchange, where the chitosan hydrochloride in the core undergoes a deprotonated crosslinking and further strengthens the gel strength of the scaffold under action of the Tris-HCl buffer; and
(7) construction of a microbial film exposed at an air-liquid interface: purging the model of the biofilm-like film by high-purity air at a flow rate of 0.4-0.8 L/min for 4-8 min so that the film layer rapidly shrinks to a hydrogel film with a thickness of no more than 50 μm due to a greater dehydration rate of the sodium alginate as compared to that of the mixed hydrogel of the scaffold, to compel some microorganisms immobilized therein to be exposed at the air-liquid interface.

2. The preparation method of claim 1, wherein the hydrogel material in step (1) is selected from gelatin, agar, agarose, and carrageenan; and the calcium ion reagent is selected from calcium chloride, calcium sulfate, and calcium citrate.

3. The preparation method of claim 1, wherein during the construction of the hydrogel precursor for microbial culture in step (3), a microbial load of the model of the biofilm-like film is controlled by adjusting an immersion time of the scaffold.

4. The preparation method of claim 1, wherein the microorganisms immobilized on an in vitro microbial model of biofilm-like film are in three states:
(1) microorganisms are exposed and survive in the air-liquid interface state by virtue of a three-dimensional porous structure of the calcium alginate so that the microorganisms are directly exposed to substances in the air or obtain water and nutrients released from the scaffold in this state;
(2) although a shell shrinks into a film, some immobilized microorganisms still survive in a hydrogel environment similar to a liquid environment, ensuring their long-term survival; and these microorganisms are indirectly exposed to soluble substances in the air by diffusion; and
(3) some immobilized microorganisms are exposed to or penetrate the polyelectrolyte layer due to proliferation or pressure caused by the shell shrinkage so that the microorganisms die immediately.

5. A three-dimensional model of a microbial film exposed at an air-liquid interface obtained by the preparation method of claim 1, also known as an in vitro microbial model of biofilm-like film, having a multi-layered structure which specifically comprises: a core, configured to be a hydrogel material containing gradually releasable nutrients and serving as a scaffold for carrying other structures; a biofilm-like film, configured to be a layer of highly biocompatible hydrogel film attached to a surface of the core and wrapping microorganisms, enabling the microorganisms to form a biofilm-like film structure on the surface of the scaffold and survive and proliferate normally; after being blown with pure air for a certain time, due to differences in swelling and dehydration rates between the core material and the surface hydrogel film material, the microorganisms are compelled to inhabit in a unique air-liquid interface state; and a polyelectrolyte layer, configured to be an extremely thin antimicrobial polyelectrolyte layer between the biofilm-like film structure and the core structure, allows substances in the core to be released into the biofilm-like film structure while preventing the microorganisms on the film from migrating to the more nutritive core material, ensuring uniform distribution of the microorganisms on the surface of the core and maintaining the microorganisms continuously exposed at the air-liquid interface; wherein the microorganisms surviving on the biofilm-like film receive nutrients stably released from the core scaffold from an inner surface of the film so that the microorganisms on the biofilm-like film can be detached from a culture medium and exposed to the air, and still survive and maintain normal biological functions.

6. The three-dimensional model of a microbial film exposed at an air-liquid interface of claim 5, wherein the scaffold is made into any three-dimensional structure and set to any volume as required, and the core of the model of the biofilm-like film containing nutrients is 5-2,000 times larger in volume than the biofilm-like film structure.

7. A preparation method for a hydrogel bead containing microorganisms immobilized at an air-liquid interface in an exposed state, comprising:
(1) construction of a hydrogel precursor of a core: first, sealing a non-biotoxic and low-temperature congealable hydrogel material in its dry state, and then performing high-temperature and high-pressure sterilization at 120° C. to 131° C. for 18-24 min;
subsequently, swelling the sterile hydrogel material in a culture medium required for immobilizing target microorganisms for 15-45 min, dissolving the hydrogel material at 55° C. to 80° C., and maintaining the hydrogel material in its sealing state at 50° C. to 70° C. for later use; dissolving chitosan hydrochloride in a non-biotoxic reagent solution that is soluble or sparingly soluble in water and capable of releasing calcium ions, and performing sterilization by filtering with a 0.22 μm sterile nylon filter head; mixing the prepared hydrogel solution with the chitosan hydrochloride solution in a mass ratio to finally ensure that an obtained mixture contains 8-12% of the hydrogel material, 1-1.5% of the chitosan hydrochloride, 1-1.5% of the calcium ion reagent, and sufficient nutrients;
(2) construction of a hydrogel precursor of a shell: first, sealing sodium alginate in its dry state, and then performing high-temperature and high-pressure sterilization at 120° C. to 131° C. for 18-22 min; subsequently, adding the sterile sodium alginate to a sterile Tris-HCl buffer of 4-6 mmol/L containing salt ions required for immobilizing target microorganisms to finally ensure that an obtained mixture contains 0.6-0.8% of the sodium alginate;
(3) culture of immobilized microorganisms: culturing the target microorganisms in a microorganism solution state for 14-18 h, and performing low-temperature centrifugation at 5000-7000 rpm at 2° C. to 8° C. for 5-10 min; washing the microorganisms collected by centrifugation with a sterile Tris-HCl buffer of 4-6 mmol/L three times, mixing the microorganisms uniformly into the hydrogel precursor of the shell, and performing vortex mixing for 1-3 min to form a shell composed of highly biocompatible sodium alginate, which combines with an appropriate buffer system and salt ions to ensure normal survival of the microorganisms in a short period of time;
(4) self-assembly of the hydrogel bead: suctioning the hydrogel precursor of the core maintained at 50° C. to 70° C. by a peristaltic pump at a rate of 1-2 ml/min, and keeping a pipeline of the peristaltic pump in an attemperator set at 50° C. to 70° C. throughout the process to prevent the hydrogel precursor of the core from premature solidification due to cooling; slowly and evenly adding the suctioned hydrogel precursor of the core dropwise to the hydrogel precursor of the shell containing the immobilized microorganisms from a height of 10-20 cm above a liquid surface of the hydrogel precursor of the shell, wherein the chitosan hydrochloride as a cationic polymer interacts with the sodium alginate as an anionic polymer to form a polyelectrolyte layer that quickly envelopes the hydrogel precursor of the core in a liquid state to create a liquid sphere structure to prevent the escape of remaining gel components; subsequently, Ca²⁺ in the hydrogel precursor of the core is gradually releasing into the hydrogel precursor of the shell to gradually form a calcium-alginate hydrogel shell around the liquid sphere, thus immobilizing the microorganisms in the hydrogel precursor of the shell; wherein there is a strong linear positive correlation between a shell thickness and self-assembly time within a certain period of time, with the shell thickness varying at a rate of 50 μm/min; and producing the hydrogel beads with different initial particle thicknesses by controlling the self-assembly time;
(5) stabilization of the hydrogel bead: stabilizing the self-assembled hydrogel bead in a sterile Tris-HCl buffer of 4-6 mmol/L containing nutrients required for the immobilized microorganisms at 2° C. to 8° C. for 5-15 min, thus facilitating replenishment of nutrients in the core under action of material exchange, and promoting cooling and solidification of gelatin in the core in the low-temperature environment to form a solid hydrogel core, where the chitosan hydrochloride in the core undergoes a deprotonated crosslinking to form an interpenetrating polymer network structure with the gelatin under action of the Tris-HCl buffer; and
(6) shrinkage of the shell of the hydrogel bead: completing initial shrinkage of the shell of the hydrogel bead with the core swelling due to a higher swelling rate of the gelatin compared to that of the calcium alginate during the stabilization; and purging the stabilized hydrogel bead by high-purity air at a flow rate of 0.4-0.8 L/min for 4-8 min so that the shell quickly shrinks to a hydrogel film with a thickness of no more than 50 μm due to a greater dehydration rate of the calcium alginate compared to that of the gelatin to compel some microorganisms immobilized therein to be exposed at the air-liquid interface.

8. The preparation method of claim 7, wherein the hydrogel material in step (1) is selected from gelatin, agar, agarose, and carrageenan; and the calcium ion reagent is selected from calcium chloride, calcium sulfate, and calcium citrate.

9. The preparation method of claim 7, wherein during the self-assembly of the hydrogel bead to prepare the hydrogel precursor of the shell containing the immobilized microorganisms in step (4), an amount of the microorganisms immobilized on the hydrogel bead is controlled by adjusting an amount of microorganisms added to the hydrogel precursor of the shell and the self-assembly time of the hydrogel bead.

10. The preparation method of claim 7, wherein the self-assembled hydrogel bead is immersed in a nutrient solution containing 40% glycerol for 20-40 min and stored at −20° C.; the cryopreserved hydrogel bead is stabilized in a sterile Tris-HCl buffer of 4-6 mmol/L containing nutrients required for the immobilized microorganisms for 20-40 min, and then incubated in a constant-temperature incubator for 2-3 h; and the hydrogel bead is purged by high-purity air at a flow rate of 0.5 L/min for 5 min to re-shrink the shell.

11. A hydrogel bead obtained by the preparation method of claim 7, having a multi-layered core-shell structure, with a core configured to be a hydrogel material containing gradually releasable nutrients, a shell configured to be a highly biocompatible hydrogel film enabling microorganisms to survive and proliferate thereon, after immersion in nutrient solution and purging with air for some time, the shell shrinks to a layer of film due to differences in swelling and dehydration rates between the core hydrogel material and the shell hydrogel material, to compel microorganisms inhabit a unique air-liquid interface state after immersion in nutrient solution and purging with air for a period of time; and an extremely thin antimicrobial polyelectrolyte layer between the core and the shell of the bead, which allows an exchange of substances including nutrients between the core and the shell while preventing the microorganisms from migrating to a more nutritive core.

12. The hydrogel bead of claim 11, wherein a volume of the core and a volume of the shell satisfy the following relationship:
$$\frac{{v}_{s p h e r e}}{{v}_{s h e l l}} = \frac{{d}^{3}}{3 {d}^{2} h - 3 d {h}^{2} + {h}^{3}}$$
wherein d is a diameter of the hydrogel bead, and h is a thickness of the layer before shrinkage.
